(19) United States
(12) Patent Application Publication
(10) Pub. No.: US 2011/0091408 A1
(43) Pub. Date: Apr. 21, 2011

(54) METHODS OF USING FLUOROALKYL PHOSPHATE COMPOSITIONS

(75) Inventor: Anilkumar Raghavanpillai, Wilmington, DE (US)

(73) Assignee: E. I. DU PONT DE NEMOURS AND COMPANY, Wilmington, DE (US)

(21) Appl. No.: 12/582,191

(22) Filed: Oct. 20, 2009

(51) Int. Cl. A61K 31/74; C10L 1/26; C09K 3/14; C25D 5/00; C09K 13/00; C09K 3/00; C23F 11/00; G03F 7/00; C09K 8/24; C09K 8/60; C10M 137/04; C11D 3/00; C11D 1/34; C08K 5/52

(52) U.S. Cl. 424/78.08; 44/379; 51/298; 205/80; 252/79.1; 252/389.21; 252/389.23; 430/331; 507/117; 507/219; 508/424; 508/433; 510/347; 510/467; 510/513; 524/127; 524/149

(57) ABSTRACT

Fluoroalkyl phosphates containing a tertiary carbon and a nonfluorinated chain are useful as surfactants and additives. The f can be used to alter a surface property of a medium and to provide resistance to blocking, open time extension, or oil repellency to a substrate.

### FIELD OF THE INVENTION

This invention relates to the field of methods of using fluoroalkyl phosphates containing a tertiary carbon and a nonfluorinated chain as surfactants and additives for coating compositions or as treatment agents to impart various surface properties to substrates

### BACKGROUND

Polyfluorinated compositions are used in the preparation of a wide variety of surface treatment materials. These polyfluorinated compositions are typically made of perfluorinated carbon chains connected directly or indirectly to nonfluorinated functional groups such as hydroxyl groups, carboxylic acid groups, and halide groups. Various compositions made from perfluorinated compounds or polymers are known to be useful as surfactants or treating agents to provide surface effects or to alter surface properties of substrates. Surface properties and effects include repellency to moisture, soil, and stains, and other effects, which are particularly useful for fibrous substrates and other substrates such as hard surfaces. Many such surfactants and treating agents are fluorinated polymers or copolymers.

Most commercially available fluorinated polymers useful as treating agents for altering surface properties of substrates contain predominantly eight or more carbons in the perfluoroalkyl chain to provide the desired properties. However, polymers containing shorter chain perfluoroalkyls have traditionally not been successful commercially for providing surface properties to treated substrates.

It is desirable to improve particular surface properties and to increase the fluorine efficiency; i.e., boost the efficiency or performance of treating agents so that lesser amounts of the expensive fluorinated composition are required to achieve the same level of performance, or so that better performance is achieved using the same level of fluorine. It is desirable to reduce the chain length of the perfluoroalkyl groups thereby reducing the amount of fluorine present, while still achieving the same or superior surface properties.

There is a need for methods to significantly improve the repellency and stain resistance of fluorinated treating agents for substrates while using lower levels of fluorine using compositions with better fluorine efficiency.

### SUMMARY OF THE INVENTION

One aspect of the present invention is a method of altering a surface property of a medium comprising adding to the medium a composition comprising one or more compounds of Formula I or Formula II:

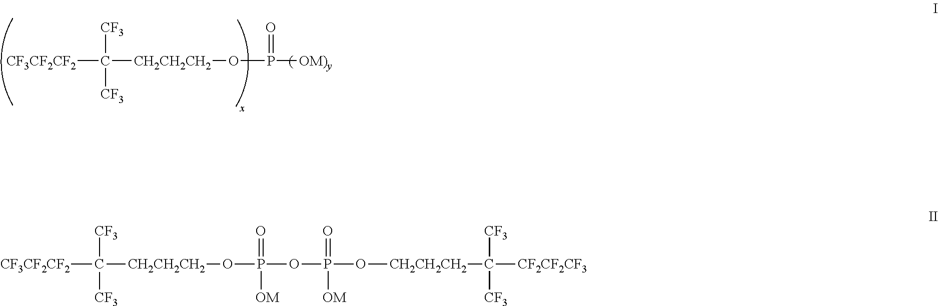

wherein x is 1 or 2, y is 1 or 2, x+y=3, and M is hydrogen, ammonium, alkali metal, or alkaline earth metal.

Another aspect of the present invention is a method of providing resistance to blocking, open time extension, or oil repellency to a substrate having deposited thereon a coating composition, comprising adding to the coating composition prior to deposition on the substrate a composition comprising one or more compounds of Formula I or Formula II, and a substrate treated according to the method.

### DETAILED DESCRIPTION

Disclosed herein is a method of altering a surface property of a medium comprising adding to the medium a composition comprising one or more compounds of Formula I or Formula II:

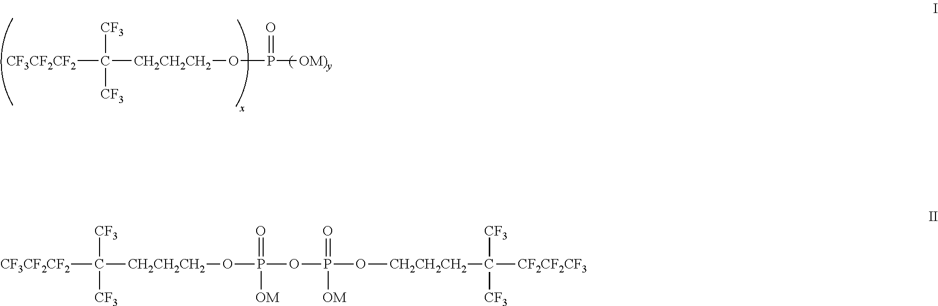

wherein x is 1 or 2, y is 1 or 2, with the proviso that x+y=3, and M is hydrogen, ammonium, alkali metal, or alkaline earth metal.

The fluoroalkyl phosphate compounds of Formula I or Formula II are useful for altering surface properties or lowering surface tension, and can be used in a variety of applications, such as coatings, cleaners, oil fields, and many other applications. The compositions are also useful in many applications involving wetting, leveling, antiblocking, foaming, and the like. The compositions have a three carbon hydrocarbon bridge between the tertiary carbon and phosphate group, allowing lower levels of fluorine while still providing good surface properties.

In one embodiment, M is ammonium or sodium. Typically M is ammonium.

In other embodiments, x is 1 and y is 1, or x is 2 and y is 1, or x is 1 and y is 2, or x is 2 and y is 2.

The compounds can be synthesized from hexafluoropropylene dimer, (F₃C)₂C═C(CF₂CF₃)F, via the following reaction scheme:

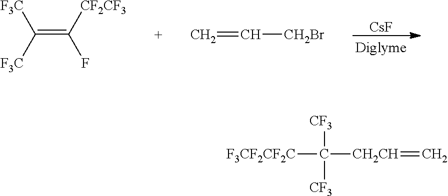

The reaction is typically run under an inert atmosphere, such as nitrogen. The desired product, 3-(perfluoro-1,1-dimethylbutyl)-1-propene, can be separated and purified using any methods known in the art, such as distillation.

The product is next reacted with borane-triethylamine, heated at 175° C. to isomerize the borane mixture, and oxidized using NaOH and H₂O₂ to produce a mixture of the primary and secondary alcohols in 4:1 ratio:

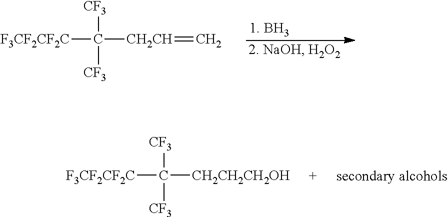

The desired primary alcohol can be separated and purified using methods known in the art, such as distillation.

The alcohol is next reacted with P₂O₅ and the resulting mixture neutralized with a base, such as the hydroxide of a particular desired cation, to form a mixture of compounds of Formula I and II. The compounds can be separated or purified by any method known in the art, or utilized as a mixture.

In one embodiment, the compositions disclosed herein have a surface tension at the critical micelle concentration (CMC) of about 20.5 mN/m or lower at a concentration of 0.0088% or higher by weight in water.

The CMC is defined as the concentration of surfactants above which micelles are spontaneously formed, at which increased concentrations of surfactant essentially no longer reduce the surface tension. The measurement and theory behind the CMC can be found, for example, in Ullmann's Encyclopedia of Industrial Chemistry, Surfactants, Kurt Kosswig, Jun. 15, 2000, Wiley-VCH Verlag GmbH & Co. KGaA (DOI: 10.1002/14356007.a25_{—}747).

In another embodiment, the compositions further comprise:
A) one or more of an agent providing at least one surface property selected from the group consisting of stain repellency, stain release, soil repellency, soil release, water repellency, oil repellency, odor control, sun protection and antimicrobial; and
B) one or more of a surfactant, pH adjuster, leveling agent, or wetting agent.

Other additives commonly used with such treating agents or finishes can also be present. Examples of such finishes or agents include processing aids, foaming agents, lubricants, anti-stains, and the like.

The surface property is typically surface tension, to provide lower critical micelle concentration (CMC) values in a variety of applications, such as coating, cleaners, oil fields, and many other applications. The method is useful in many applications involving processes such as, for example, wetting, leveling, antiblocking, foaming, penetration, spreading, flowing, emulsification and dispersion stabilization. Types of surface properties which can be altered include wetting, penetration, spreading, leveling, flowing, emulsifying, dispersing, repelling, releasing, lubricating, etching, bonding, and stabilizing.

Other additives commonly used with such treating agents or finishes can also be present in the composition, such as surfactant, pH adjuster, leveling agent, wetting agent, processing aids, foaming agents, lubricants, anti-stains, and the like.

Types of medium which can be used in the methods disclosed herein include a coating composition, latex, polymer, floor finish, ink, emulsifying agent, foaming agent, release agent, repellency agent, flow modifier, film evaporation inhibitor, wetting agent, penetrating agent, cleaner, grinding agent, electroplating agent, corrosion inhibitor, etchant solution, soldering agent, dispersion aid, microbial agent, pulping aid, rinsing aid, polishing agent, personal care composition, drying agent, antistatic agent, floor finish, or bonding agent.

Also provided, in another embodiment, is a method of providing resistance to blocking, open time extension, or oil repellency to a substrate having deposited thereon a coating composition comprising adding to the coating composition prior to deposition on the substrate a composition comprising Formula I, Formula II, or a mixture thereof, as described above.

The term “blocking” is used herein to mean the undesirable sticking together of two coated surfaces when pressed together, or placed in contact with each other for an extended period of time. When blocking occurs separation of the surfaces can result in disruption of the coating on one or both surfaces. Thus improved resistance to blocking is beneficial in many situations where two coated surfaces need to be in contact, for example on window frames.

The term “open time extension” is used herein to mean the time during which a layer of liquid coating composition can be blended into an adjacent layer of liquid coating composition without showing a lap mark, brush mark, or other application mark. It is also called wet-edge time. Latex paint containing low boiling volatile organic chemicals (VOC) has shorter than desired open-time due to lack of high boiling temperature VOC solvents. Lack of open time extension will cause surface defects such as overlapping brush marks or other marks. A longer open time extension is beneficial when the appearance of the coated surface is important, as it permits application of the coating without leaving overlap marks, brush marks, or other application marks at the area of overlap between one layer of the coating and an adjacent layer of the coating.

A compound of Formula I, Formula II, or a mixture thereof, is typically introduced into a coating composition by thoroughly stirring the compound into the coating composition at ambient temperature. More elaborate mixing can be employed such as using a mechanical shaker or providing heat or other methods.

Suitable substrates include porous, fibrous or hard surface substrates. Specific examples of suitable substrates include wood, paper, leather, stone, masonry, mineral surfaces concrete, unglazed tile, brick, porous clay, granite, limestone, grout, mortar, marble, wood, gypsum board, terrazzo, glass, composite materials such as terrazzo, and wall and ceiling panels including those fabricated with gypsum board. Many suitable substrates are used in statuary, monuments, and in the construction of buildings, roads, parking ramps, driveways, floorings, fireplaces, fireplace hearths, counter tops, and decorative uses in interior and exterior applications.

The coating composition is typically applied by contacting the substrate with the coating composition by conventional methods, such as, for example, brush, spray, roller, doctor blade, wipe, immersion, dip techniques, foam, liquid injection, casting. Optionally, more than one application can be used, particularly on porous surfaces.

Suitable coating compositions, also known by the term “coating base”, include compositions containing, typically a liquid formulation, of an alkyd coating, Type I urethane coating, unsaturated polyester coating, or water-dispersed coating that can be applied to a substrate for the purpose of creating a lasting film on the substrate surface, such as a paint or a stain.

The term “alkyd coating” as used herein means a conventional liquid coating based on alkyd resins, typically a paint, clear coating, or stain. The alkyd resins are complex branched and cross-linked polyesters containing unsaturated aliphatic acid residues. Conventional alkyd coatings utilize, as the binder or film-forming component, a curing or drying alkyd resin. Alkyd resin coatings contain unsaturated aliphatic acid residues derived from drying oils. Alkyd resins spontaneously polymerize in the presence of oxygen or air to yield a solid protective film. The polymerization is termed “drying” or “curing” and occurs as a result of autoxidation of the unsaturated carbon-carbon bonds in the aliphatic acid component of the oil by atmospheric oxygen. When applied to a surface as a thin liquid layer of formulated alkyd coating, the cured films that form are relatively hard, non-melting, and substantially insoluble in many organic solvents that act as solvents or thinners for the unoxidized alkyd resin or drying oil. Such drying oils have been used as raw materials for oil-based coatings and are described in the literature.

The term “urethane coating” as used herein means a conventional liquid coating based on Type I urethane resins, typically a paint, clear coating, or stain. Urethane coatings typically contain the reaction product of a polyisocyanate, usually toluene diisocyanate, and a polyhydric alcohol ester of drying oil acids. Urethane coatings are classified by ASTM D-1 into five categories. Type I urethane coatings contain a pre-reacted autoxidizable binder as described in Surface Coatings Vol. I, previously cited. Type I urethane coatings are also known as uralkyds, urethane-modified alkyds, oil-modified urethanes, urethane oils, or urethane alkyds, are the largest volume category of polyurethane coatings which include paints, clear coatings, or stains. A cured coating is formed by air oxidation and polymerization of the unsaturated drying oil residue in the binder.

The term “unsaturated polyester coating” as used herein means a conventional liquid coating based on unsaturated polyester resins, dissolved in monomers and containing initiators and catalysts as needed, typically as a paint, clear coating, or gel coat formulation. Unsaturated polyester resins contain as an unsaturated prepolymer the product obtained by condensation polymerization of a glycol such as 1,2-propylene glycol or 1,3-butylene glycol with an unsaturated acid such as maleic (or of maleic and a saturated acid, e.g., phthalic) in the anhydride form. The unsaturated prepolymer is a linear polymer containing unsaturation in the chain. The unsaturated prepolymer is dissolved in a suitable monomer, for instance styrene, to produce the resin. The resulting film that is deposited on the substrate is produced by copolymerization of the linear polymer and monomer by means of a free radical mechanism. Such coating compositions are frequently termed “gel coat” finishes. For curing coatings at room temperature, the decomposition of peroxides into free radicals is catalyzed by certain metal ions, usually cobalt. The unsaturated polyester resins that cure by a free radical mechanism are also suited to irradiation curing using, for instance, ultraviolet light. Irradiation curing, in which no heat is produced, is particularly suited to films on wood or board. Other radiation sources, for instance electron-beam curing, are also used.

The term “water-dispersed coatings” as used herein means coatings intended for the decoration or protection of a substrate where water is an essential dispersing component such as an emulsion, latex, or suspension of a film-forming material dispersed in an aqueous phase. “Water-dispersed coating” is a general classification that describes a number of formulations and includes members of the above described coatings as well as other coatings. Water-dispersed coatings in general contain other common coating ingredients. Examples of water-dispersed coatings include pigmented coatings such as latex paints, unpigmented coatings such as wood sealers, stains, and finishes, coatings for masonry and cement, and water-based asphalt emulsions. A water dispersed coating optionally contains surfactants, protective colloids and thickeners, pigments and extender pigments, preservatives, fungicides, freeze-thaw stabilizers, antifoam agents, agents to control pH, coalescing aids, and other ingredients. Latex paints contain a film forming material, which is a latex polymer of acrylate, acrylic, vinyl-acrylic, vinyl, or a mixture thereof. Such water-dispersed coating compositions are described by C. R. Martens in “Emulsion and Water-Soluble Paints and Coatings” (Reinhold Publishing Corporation, New York, N.Y., 1965).

Another embodiment provides a substrate treated according to the method described above. The substrate can be any suitable porous nonporous, fibrous or hard surface substrates, as recited hereinabove.

The fluoroalkyl phosphate compositions and methods disclosed herein can be useful in a variety of applications where a low surface tension is desired, such as coating formulations for glass, wood, metal, brick, concrete, cement, natural and synthetic stone, tile, synthetic flooring, paper, textile materials, plastics, and paints. The fluoroalkyl phosphate compositions are useful in waxes, finishes, and polishes to improve wetting, leveling, and gloss for floors, furniture, shoe, and automotive care. The compositions are useful in a variety of aqueous and non-aqueous cleaning products for glass, tile, marble, ceramic, linoleum and other plastics, metal, stone, laminates, natural and synthetic rubbers, resins, plastics, fibers, and fabrics.

The fluoroalkyl phosphate compositions and methods disclosed herein are further suitable for use in agricultural compositions. They can be used as wetting agents for compositions containing herbicides, weed killers, hormone growth regulators, parasiticides, insecticides, germicides, bactericides, nematocides, microbiocides, defoliants or fertilizers, therapeutic agents, antimicrobials: as a wetting agent for foliage, for live stock dips and to wet live stock skins; and as an ingredient in sanitizing, discoloring and cleaning compositions, and in insect repellent compositions.

The fluoroalkyl phosphate compositions and methods disclosed herein are also suitable for the use in compositions for fluorochemical blood substitutes, textile treatment baths, fiber spin finishes, personal care products (including like shampoos, conditioners, creams, rinses), cosmetic products for the skin (such as therapeutic or protective creams and lotions, oil and water repellent cosmetic powders, deodorants and antiperspirants), nail polish, lipstick, toothpastes, fabric care products (such as stain pretreatments and/or stain removers for clothing, carpets and upholstery), laundry detergents, rinse-aid (for car washes and in automatic dishwashers).

The fluoroalkyl phosphate compositions and methods disclosed herein are further suitable for use in the petroleum industry as a wetting agent for oil well treatments (including drilling muds and additives to improve tertiary oil well recovery, as well as in extreme pressure lubricants and as a lubricating cuffing oil improver, to improve penetration times); as a film evaporation inhibitor for gasoline, jet fuel, solvents, hydrocarbons; as a lubricant or cutting oil improver to improve penetration times; as an oil spill collecting agent; and as oil well stimulation additives.

The fluoroalkyl phosphate compositions and methods disclosed here in are further suitable for the use in writing inks, printing inks, photography developer solutions, fighting forest fires, dry chemical fire extinguishing agents, aerosol-type fire extinguishers, thickening agents to form gels for solidifying or encapsulating medical waste, and photoresists, developers, cleaning solutions, oxide etching compositions, developers, polishers, and resist inks in the manufacturing, processing, and handling of semiconductors and electronics.

The fluoroalkyl phosphate compositions and methods disclosed herein are further suitable for the use in textile and leather industries as a wetting agent, antifoaming agent, penetrating agent or emulsifying agent; or as a lubricant for textiles, nonwoven fabrics and leather treatment; for fiber finishes for spreading, and uniformity; as a wetting agent for dyeing; as a binder in nonwoven fabrics; and as a penetration additive for bleaches.

The fluoroalkyl phosphate compositions and methods disclosed herein are further suitable for the use in the mining and metal working industries, in the pharmaceutical industry, automotives, building maintenance and cleaning, in household, cosmetic and personal products, and in photography and graphic arts to provide improved surface effects.

The fluoroalkyl phosphate compositions and methods disclosed herein can be incorporated into products that function as antifogging agents for glass surfaces and photography films, and as antistatic agent for magnetic tapes, phonograph records, floppy disks, disk drives, rubber compositions, PVC, polyester film, photography films, and as surface treatments for optical elements (such as glass, plastic, or ceramic beads).

The fluoroalkyl phosphate compositions and methods disclosed herein are also useful as foam control agents in polyurethane foams, spray-on oven cleaners, foamed kitchen and bathroom cleansers and disinfectants, aerosol shaving foams, and in textile treatment baths.

The fluoroalkyl phosphate compositions and methods disclosed herein are useful as emulsifying agents for polymerization, particularly of fluoromonomers, as latex stabilizers, as mold release agents for silicones, photoemulsion stabilizers, inorganic particles, and pigments.

The compositions and methods disclosed herein provide several unexpected advantages. The compounds are not prepared by electrochemical fluorination or telomerization, providing the ability to prepare previously difficult to synthesize compounds having odd number of carbons in the chain. The formation of large amounts of impurities are avoided and the environmental footprint is minimized. The compositions are more fluorine efficient than typical telomerization derived products. The lower level of fluorine present in the present compositions is more economical, but provides equivalent or superior performance to conventional surfactants containing higher levels of fluorine.

### EXAMPLES

All solvents and reagents, unless otherwise indicated, were purchased from commercial sources (Alfa Aesar, Ward Hill Mass., TCI America Organic Chemicals, Portland Oreg.) and used directly as supplied. Phosphorus pentoxide was obtained from Sigma Aldrich, Milwaukee, Wis. Perfluoro-2-methyl-2-pentene (hexafluoropropene-dimer) was obtained from Oakwood Products Inc, W Columbia, S.C.

### Test Method 1

### Surface Tension Measurements

The surface tension measurements of the surfactants were measured in fresh Millipore filtered water using the Wilhelmy plate method on an automated Krüss tensiometer (Model K11, Krüss USA, Nazareth, Pa.) or a Sigma70 tensiometer (KSV Instruments Inc., Monroe, Conn.) used in accordance with the manufacturers' manuals. Millipore filters are available from Millipore Corporation, Billerica, Mass.

A clean, dry 50 mL plastic beaker was filled approximately 40 mL of the desired solution for surface tension measurement. The beaker was placed on the sample platform of the Kruss K11 tensiometer. The platinum surface tension probe was removed from the tensiometer hook and rinsed with deionized water and dried with the blue part of the flame from the propane torch. The probe was then air cooled and reinserted onto the tensiometer hook. The surface tensions of the desired solutions were as described in the Kruss K11 tensiometer operating manual.

To determine Critical Micelle Concentration (CMC), the surface tension was measured as a function of surfactant concentration. Surface tension was then plotted vs. log concentration. The resulting curve had a nearly horizontal portion at concentrations higher than the CMC and had a negative steep slope at concentrations less than the CMC. The CMC was calculated as that concentration of the curve where the flat portion and the extrapolated steep slope intersected. The Surface Tension beyond CMC was the value in the flat portion of the curve. The CMC should be as low as possible to provide the lowest cost for effective performance.

### Test Method 2

### Surface Tension Measurements in HCl and KCl for Oil Field

Solutions of 2% KCl and 15% HCl in water were typically used in the surface tension measurements for oilfield applications because they mimic the stimulation fluid types that are pumped down hole into wells. The 2% KCl solution was similar to the salinity of the fracture fluids that are used to hydraulically fracture a well. The 15% HCl solution emulated the acidizing stimulation treatment fluid that is used to help dissolve the formation rock in wells.

A 1 wt % stock solution was prepared for the fluorosurfactant to be analyzed in 2% KCl water, or 15% HCl water depending on the desired oilfield application for which the surface tension was being measured. The stock solution was stirred overnight (for approximately 12 hours) to ensure complete mixing. Additional concentrations of the fluorosurfactant for analysis were made by diluting the stock solution in order to formulate the final sample. The concentration dilution samples were shaken thoroughly and then left to sit undisturbed for 30 minutes. The surface tension of these samples was measured using a Kruss Tensiometer, K11 Version 2.501 in accordance with instructions with the equipment. The Wilhelmy Plate method was used. A vertical plate of known perimeter was attached to a balance, and the force due to wetting was measured. 10 replicates were tested of each dilution, and the following machine settings were used:

Method: Plate Method SFT

Interval: 1.0 s

Wetted length: 40.2 mm

Reading limit: 10

Min Standard Deviation: 2 dynes/cm

Gr. Acc.: 9.80665 m/s²

Lower surface tension indicated superior performance.

### Test Method 3

### Wetting and Leveling Test

The wetting and leveling ability of the samples was tested by adding each sample to a floor polish (RHOPLEX™ 3829, Rohm & Haas, Spring House, Pa.) and applying the mixture to half of 12 inch×12 inch (30.36 cm×30.36 cm) vinyl tile stripped with a Comet cleanser. A 1 wt % solution of the surfactant to be tested was prepared by dilution with deionized water. Following the manufacturer protocols, a 100 g portion of the RHOPLEX™ 3829 formulation was prepared, followed by addition of 0.75 g of the 1 wt % surfactant solution, to provide a test floor polish.

The test floor polish was applied to a tile by placing a 3 mL portion of the test polish in the center of the tile, spreading the solution from top to bottom using an applicator, and finally placing a large “X” across half of the tile, using the applicator. The tile was allowed to dry for 30 min. A total of 5 coats was applied. After each coat, the tile was rated on a 1 to 5 scale (1 being the worst, 5 the best) on the surfactant's ability to promote wetting and leveling of the polish on the tile surface. The rating was determined based on comparison of a tile treated with the floor polish that contained no fluorosurfactant or leveling aids according to the following scale:

Subjective Tile Rating Scale

1 Uneven surface coverage of the film, significant streaking and surface defects

2 Visible streaking and surface defects, withdrawal of the film from the edges of the tile

3 Numerous surface defects and streaks are evident but, generally, film coats entire tile surface

4 Minor surface imperfections or streaking

5 No visible surface defects or streaks

### Preparation of 3-(Perfluoro-1,1-dimethylbutyl)-1-propene [CF₃CF₂CF₂C(CF₃)₂CH₂CH═CH₂]

3-(perfluoro-1,1-dimethylbutyl)-1-propene was synthesized by a modified procedure described in the literature (Dmowski, W and Woznjacki, R J. Fluorine. Chem., 36, 1987, 385-394).

A 4 necked 250 mL flask fitted with a dry ice condenser, septa and temperature probe, dropping funnel, stirring device was charged with dry diglyme (60 mL), anhydrous cesium fluoride (25.2 g) and perfluoro-2-methyl-2-pentene (50.0 g) (or mixture of perfluoro-2-methyl-2-pentene and perfluoro-4-methyl-2-pentene) under nitrogen atmosphere. The mixture was heated at 35° C. and with vigorous stirring added allyl bromide (20.14 g). The mixture was heated at 40° C. for 36 h and contents were fractionally distilled. The fractions collected at 70-90° C. and 90-130° C. were mainly the desired product. The fractions were combined and redistilled using a fractionating column to obtain pure product 3-(perfluoro-1,1-dimethylbutyl)-1-propene (50.37 g) Bp. 107-113° C.

### Preparation of 3-(perfluoro-1,1-dimethylbutyl)-1-propanol [CF₃CF₂CF₂C(CF₃)₂CH₂CH₂CH₂OH]

This compound was synthesized by a modified procedure described in the literature (Dmowski, W, Plenkiewicz, H.; Porwisiak, J. J. Fluorine. Chem., 41, 1988, 191).

A 3-necked flask fitted an addition rubber septa reflux condenser and addition funnel was assembled while hot and flushed with nitrogen. To the flask was added borane-triethylamine (1.60 g, 13.9 mmol) via a syringe followed by 3-(perfluoro-1,1-dimethylbutyl)-1-propene via the addition funnel over 5 minutes. The reaction mixture hated at 75° C. for 3 h and then the triethylamine was distilled off. The crude mixture was analyzed by GC and showed a mixture of 1° and 2° boranes in 1:2.3 ratio. The residue was heated at 175° C. for 16 h and the GC analysis of the mixture indicates a 4:1 mixture of 1° and 2° boranes along with traces of fluorinated products resulting from dimerization. The crude borane mixture was then diluted with THF (10 mL) and added a solution of 30% H₂O₂ (3.78 mL), water (0.25 mL) and NaOH (0.3 g) at 0° C. The reaction was allowed to stir at RT for 24 h. 40 mL of ether was added, the organic phase separated and washed successively with water (30 mL) and dried over anhydrous MgSO₄. GC analysis of the mixture indicates a 4:1 mixture of 1° and 2° alcohols along with traces of unidentified products. Removal of the solvent followed by careful distillation produced the desired primary alcohol (CF₃CF₂CF₂C(CF₃)₂CH₂CH₂CH₂OH) in 98% purity (6.4 g, 16.9 mmol, 61%) bp: 86-87° C. @ 20 mm Hg.

### Example 1

### (CF₃CF₂CF₂C(CF₃)₂CH₂CH₂CH₂O-Phos)

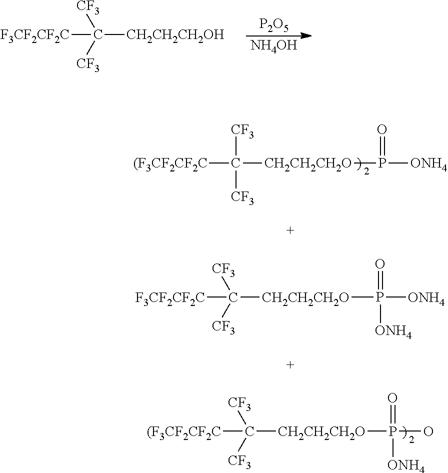

Phosphorous pentoxide (0.33 g, 2.33 mmol) was transferred to a reaction flask flushed under nitrogen atmosphere and added slowly 3-(perfluoro-1,1-dimethylbutyl)-1-propanol (2.0 g, 5.29 mmol). The mixture was then heated at 85° C. for 12 h and allowed to cool to RT. To the resulting mixture was added 2 mL isopropyl alcohol followed by 5 mL H₂O. The resulting slurry was treated with 30% NH₄OH (0.32 g) and freeze-dried to obtain a white powder comprising a complex mixture of phosphates (2.0 g). 31P NMR showed 4 phosphorous signals corresponding to the three possible esters and phosphorous impurity. The crude product was evaluated for CMC and surface tension beyond the CMC by Test Method 1.

### Example 2

### Surface Tension Measurements in Water

Surface tensions of the composition prepared in Example 1 in water were measured according to Test Method 1, and compared with commercially available Zonyl® FSP (anionic phosphate fluorosurfactant, commercial formulation with 35% fluorinated surfactant, available from E. I. du Pont de Nemours and Company, Wilmington, Del.). The results are summarized in Table 1 below.

TABLE 1
Surface Tension of example 1 and Zonyl ® FSP in water

| | | | | Surface |
|---|---|---|---|---|
| | | Surface | | tension |
| | Concentration | tension | | beyond CMC |
| Surfactant | wt % | mN/m | CMC wt % | mN/m |
| Zonyl ® FSP | 0.1 | 16.1 | | |
| (Comparative) | 0.01 | 16.9 | | |
| | 0.005 | 28.4 | 0.0091 | 16.9 |
| | 0.001 | 59.4 | | |
| | 0.0001 | 61.0 | | |
| Example 1 | 0.1 | 20.1 | | |
| | 0.01 | 20.5 | | |
| | 0.005 | 28.5 | 0.0088 | 20.5 |
| | 0.001 | 40.3 | | |
| | 0.0001 | 54.9 | | |

Example 1 showed very good critical micelle concentration (CMC) and lower surface tensions at low concentrations although it has ˜25% less fluorine present in the molecule compared to the comparative Zonyl® FSP

### Example 2

### Surface Tension Measurements in HCl and KCl

Surface tensions of the composition prepared in Example 1 in HCl and KCl were measured according to Test Method 2, and compared with commercially available Zonyl® FSH (nonionic fluorosurfactant, commercial formulation with 50% fluorinated surfactant, available from E. I. du Pont de Nemours and Company, Wilmington, Del.). available from E. I. du Pont de Nemours and Company, Wilmington, Del.) and Zonyl® FS-510 (amine oxide-based fluorosurfactant, commercial formulation with 40% fluorinated surfactant, available from E. I. du Pont de Nemours and Company, Wilmington, Del.). The results are summarized in Table 2.

TABLE 2
Surface Tension of Example 1 and comparatives in HCl and KCl

| | | Surface | Surface |
|---|---|---|---|
| | | tension | tension |
| | Concentration, | in HCl | in KCl |
| Surfactant | wt % | mN/m | mN/m |
| Zonyl ® FSH | 0.05 | 18.6 | 19.3 |
| (Comparative B) | 0.01 | 21.2 | 23.9 |
| | 0.001 | 30.8 | 40.4 |
| | 0 | 72.7 | 72.7 |
| Zonyl ® FS-510 | 0.05 | 15.8 | 15.9 |
| (Comparative C) | 0.01 | 19.0 | 16.5 |
| | 0.001 | 50.0 | 43.8 |
| | 0 | 72.7 | 72.7 |
| Example 1 | 0.05 | 26.0 | 20.4 |
| | 0.01 | 29.9 | 21.0 |
| | 0.001 | 64.4 | 23.5 |
| | 0 | 72.7 | 72.7 |

The data indicates that Example 1 showed higher surface tensions in 15% HCl at a given concentration compared to the comparative, however Example 1 has reduced fluorine content compared to the comparative. Surface tensions of example 1 in 2% KCl showed similar surface tensions to that of the standard Zonyl® FS-510 and a significantly better CMC.

### Example 3

### Tests of Wetting and Leveling Ability

This example illustrates the performance according to Test Method 3 of the compound prepared in Example 1 as wetting and leveling agents in a commercial floor polish, RHOPLEX™ 3829 (N-29-1) available from Rohm & Haas, Spring House, Pa. In a Control test, no leveling agent was added to the floor polish. Comparative experiments were also performed using commercially available Zonyl® FSO (nonionic surfactant with 50% fluoroalkyl ethoxylate surfactant, available from E. I. du Pont de Nemours and Company, Wilmington, Del.) as the wetting and leveling agent. All samples were measured at 75 ppm (microgram/g) loading, and at the same time, to nullify potential variations in room humidity and temperature. The results are listed in Table 3. A high rating indicates superior performance. Surface tensions also measured in RHOPLEX™ 3829 (N-29-1) as per Test Method 1 (Table 4).

TABLE 3
Ratings for Wetting and Leveling Test

| | Coating No. | | | | | |
|---|---|---|---|---|---|---|
| | 1 | 2 | 3 | 4 | 5 | |
| | Rating | | | | | Average |
| Control | 1 | 1 | 1 | 1 | 1 | 1 |
| Zonyl ® FSO | 1.5 | 2.5 | 3.5 | 4 | 4 | 3.1 |
| (Comparative D) | | | | | | |
| 1A | 1.5 | 2 | 3 | 3.5 | 3.5 | 2.7 |

Rating (1-5, 1 being the worst)

TABLE 4
Surface tension of Example 1 in Rhoplex ® 3829 (N-29-1)

| | Concentration | Surface tension |
|---|---|---|
| Surfactant | wt % | in RHOPLEX ™ 3829 |
| Zonyl ® FSO | 0.1 | 27.8 |
| (Comparative D) | 0.01 | 28.2 |
| | 0.001 | 30.4 |
| | 0.0001 | 32.9 |
| Example 1 | 0.1 | 25.6 |
| | 0.01 | 29.5 |
| | 0.001 | 29.1 |
| | 0.0001 | 32.8 |

The results in Table 3, indicate that the composition prepared in Example 1 reduced fluorine showed wetting and leveling characteristics slightly lower than comparative Zonyl® FSO, but significantly better than the Control sample, where no leveling agent was added.

The results in Table 4, indicate that the composition prepared in Example 1 reduced fluorine showed similar surface tensions to that of comparative Zonyl® FSO.

What is claimed is:

1. A method of altering a surface property of a medium comprising adding to the medium a composition comprising one or more compounds of Formula I or Formula II: 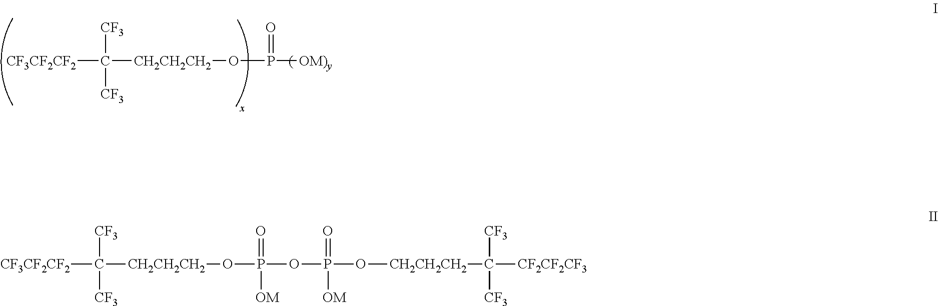
wherein x is 1 or 2, y is 1 or 2, x+y=3, and M is hydrogen, ammonium, alkali metal, or alkaline earth metal.

2. The method of claim 1 wherein M is ammonium or sodium.

3. The method of claim 1 wherein M is ammonium.

4. The method of claim 1 wherein the surface property is surface tension.

5. The method of claim 1 wherein the surface property is selected from the group consisting of wetting, penetration, spreading, leveling, flowing, emulsifying, dispersing, repelling, releasing, lubricating, etching, bonding, and stabilizing.

6. The method of claim 1 wherein the medium is a coating composition, polymer, or polymer dispersion.

7. The method of claim 1 wherein the medium is a floor finish, ink, emulsifying agent, foaming agent, release agent, repellency agent, flow modifier, film evaporation inhibitor, wetting agent, penetrating agent, cleaner, grinding agent, electroplating agent, corrosion inhibitor, etchant solution, soldering agent, dispersion aid, antimicrobial agent, pulping aid, rinsing aid, polishing agent, personal care composition, drying agent, antistatic agent, floor finish, oil well stimulation additive, or bonding agent.

8. A product made using the method of claim 1 wherein the product is a personal care item, floor finish, furniture finish, shoe finish, automotive care product, agricultural composition, cleaning product, blood substitute, textile treatment bath, fiber spin finish, laundry detergent, rinse-aid, oil well treatment, drilling muds, engine fuel, lubricant, oil spill collecting agent, ink, photography developer solution, fire extinguishing agent, or disinfectant.

9. A method of providing resistance to blocking, open time extension, or oil repellency to a substrate having deposited thereon a coating composition, comprising adding to the coating composition prior to deposition on the substrate a composition comprising one or more compounds of Formula I or Formula II: 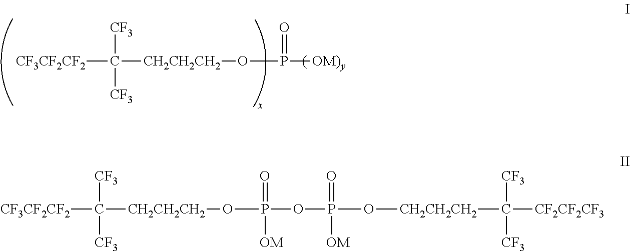
wherein x is 1 or 2, y is 1 or 2, x+y=3, and M is hydrogen, ammonium, alkali metal, or alkaline earth metal.

10. The method of claim 1 wherein M is ammonium or sodium.

11. The method of claim 7 wherein M is ammonium.

12. The method of claim 7 wherein the coating composition is an alkyd coating, Type I urethane coating, unsaturated polyester coating or water dispersed coating.

13. A substrate treated according to the method of claim 7.

14. The substrate of claim 11 which is selected from the group consisting of wood, paper, leather, stone, masonry, concrete, unglazed tile, brick, porous clay, granite, limestone, grout, mortar, marble, wood, gypsum board, terrazzo, glass, and composite materials.
